(19) United States
(12) Patent Application Publication
(10) Pub. No.: US 2012/0003229 A1
(43) Pub. Date: Jan. 5, 2012

(54) MODULATORS OF CYTOKINE MEDIATED SIGNALLING PATHWAYS AND INTEGRIN ALPHAVBETA3 RECEPTOR ANTAGONISTS FOR COMBINATION THERAPY

(76) Inventors: Herve Geneste, Neuhofen (DE); Wilfried Hornberger, Neustadt (DE)

(21) Appl. No.: 13/210,178

(22) Filed: Aug. 15, 2011

Related U.S. Application Data
(63) Continuation of application No. 11/375,305, filed on Mar. 14, 2006, now abandoned.
Application No. 11/375,305 is a continuation of application No. 10/089,723, filed on Aug. 19, 2002, now abandoned.

(30) Foreign Application Priority Data
Oct. 6, 1999 (DE) 199 48 269.1
Dec. 24, 1999 (DE) 199 62 998.6
Jun. 6, 2000 (DE) 100 27 514.1
Jun. 14, 2000 (DE) 100 28 575.9
Aug. 11, 2000 (DE) 100 39 998.3

(51) Int. Cl. A61K 39/395; A61P 37/06; A61P 29/00

(52) U.S. Cl. 424/142.1

(57) ABSTRACT

The invention relates to the use of modulators of cytokine mediated signalling pathways in combination with integrin αᵥβ₃ receptor antagonists for the treatment or prevention of diseases, particularly to the use of a pharmaceutical composition, comprising a modulator of cytokine mediated signalling pathways and an integrin αᵥβ₃ receptor antagonist, for the treatment or prevention of inflammatory or autoimmune disorders, particularly for the treatment or prevention of rheumatoid arthritis and to the pharmaceutical composition itself.

The invention relates to the use of modulators of cytokine mediated signalling pathways in combination with integrin αᵥβ₃ receptor antagonists for the treatment or prevention of diseases, particularly to the use of a pharmaceutical composition, comprising a modulator of cytokine mediated signalling pathways and an integrin αᵥβ₃ receptor antagonist, for the treatment or prevention of inflammatory or autoimmune disorders, particularly for the treatment or prevention of rheumatoid arthritis and to the pharmaceutical composition itself.

Rheumatoid arthritis (RA) is a complex chronic inflammatory disease which affects approximately 1 to 3% of the general population. A variety of anti-inflammatory and immunosuppresive regimens have been employed to limit disease. However, significant toxicity is associated with current therapies which subdue but ultimately fail to stop progression to erosive joint destruction.

It is known, that TNFα, a cytokine produced by numerous cell types, has been implicated in activating tissue inflammation and causing joint destruction in rheumatoid arthritis (see e.g., Moeller, A., et al. (1990) Cytokine 2:162-169; U.S. Pat. No. 5,231,024 to Moeller et al.; European Patent Publication No. 260 610 B1 by Moeller, A.; WO 9729131; Tracey and Cerami, supra; Arend, W. P. and Dayer, J-M. (1995) Arth. Rheum. 38:151-160; Fava, R. A., et al. (1993) Clin. Exp. Immunol. 94:261-266).

On the other hand, it is known that cytokines, for example, IL-10 and IL-4 may have an anti-inflammatory effect. Therefore, it is believed, that compounds that suppress or inhibit proinflammatory cytokine mediated signalling pathways (anti-proinflammatory-cytokine compounds) and compounds that stimulate anti-inflammatory cytokine mediated signalling pathways (anti-inflammatory compounds) may be useful for the treatment of RA (Bredveld, Rheumatology 1999, 38, 11 to 13).

Cheresh et al. describe that suppressors of angiogenesis, such as αᵥβ₃ antagonists, might be useful for the treatment of RA (The Journal of Clinical Investigation 1999, 103, 1, p.47 to 54; Braz. J. Med. Biol. Res. 1999, 32, p. 573 to 581).

It is an object of the present invention to provide an effective method of treatment or prevention of inflammatory or autoimmune disorders, particularly for the treatment or prevention of rheumatoid arthritis, with acceptable side effects and advantageous properties.

We have found that this object is achieved by using modulators of cytokine mediated signalling pathways in combination with an integrin αᵥβ₃ receptor antagonist.

By combining compounds which act as modulators of cytokine mediated signalling pathways and integrin αᵥβ₃ receptor antagonists either in one formulation or as a kit-of-parts combination by applying both separately via the same or different routes, it is possible to achieve an inhibitory effect on inflammatory pathomechanisms causing rheumatoid arthritis significantly more pronounced than one of the two treatments alone at the given doses. The combination of modulators of cytokine mediated signalling pathways and integrin αᵥβ₃ receptor antagonists in doses too low to be effective alone is as least as effective as a high mono-therapy with either agent and has less potential for side-effects than one principle alone.

Therefore, the invention relates to the use of modulators of cytokine mediated signalling pathways in combination with an integrin αᵥβ₃ receptor antagonist for the manufacture of medicaments for the treatment or prevention of diseases, particularly of inflammatory or autoimmune disorders, particularly of rheumatoid arthritis.

Inflammatory or autoimmune disorders are, for example, rheumatoid arthritis, rheumatoid spondylitis, osteoarthritis, gouty arthritis, allergy, multiple sclerosis, autoimmune diabetes, autoimmune uveitis or nephrotic syndrome.

In a preferred embodiment, the combination according to the invention can be used for the manufacture of medicaments for the treatment or prevention of rheumatoid arthritis.

Preferred modulators of cytokine mediated signalling pathways results in an anti-inflammatory effect. Therefore, according to the invention, modulators of cytokine mediated signalling pathways preferred are compounds that suppress or inhibit proinflammatory cytokine mediated signalling pathways (anti-proinflammatory-cytokine compounds),

such as, for example, TNFα-inhibitors, particularly TNFα-anti-bodies, inhibitors of interleukin-1β converting enzyme (ICE inhibitors), inhibitors of Interleukin 1 (IL-1 inhibitors) such as IL-1RA (IL-1 receptor antagonist, Synergen/Amgen), inhibitors of Interleukin 2 (IL-2 inhibitors) such as anti-IL2R antibodies, DAB 486-IL-2 and/or DAB 389-IL-2 (IL-2 fusion proteins, Seragen, see e.g., Arthritis & Rheumatism (1993) Vol. 36, 1223) or Anti-Tac (humanized anti-IL-2Ra, Protein Design Labs/Roche), inhibitors of Interleukin 6 (IL-6 inhibitors), inhibitors of Interleukin 12 (IL-12 inhibitors), inhibitors of Interleukin 17 (IL-17 inhibitors, see e.g., Arthritis & Rheumatism (1996) Vol. 39, No. 9 (supplement), S120), inhibitors of Interleukin 18 (IL-18 inhibitors) or antinflammatory or antiautoimmune drugs such as R973401 (phosphodiesterase Type IV inhibitor, see e.g., Arthritis & Rheumatism (1996) Vol. 39, No. 9 (supplement), S282), MK-966 (COX-2 Inhibitor, see e.g., Arthritis & Rheumatism (1996) Vol. 39, No. 9 (supplement), S81), Iloprost (see e.g., Arthritis & Rheumatism (1996) vol. 39, No. 9 (supplement), S82), methotrexate, thalidomide (see e.g., Arthritis & Rheumatism (1996) Vol. 39, No. 9 (supplement), 5282) and thalidomide-related drugs (e.g., Celgen), leflunomide (anti-inflammatory and cytokine inhibitor, see e.g., Arthritis & Rheumatism (1996) Vol. 39, No. 9 (supplement), S131, Inflammation Research (1996) Vol. 45, pp. 103-107), tranexamic acid (inhibitor of plasminogen activation, see e.g., Arthritis & Rheumatism (1996) Vol. 39, No. 9 (supplement), S284), T-614 (cytokine inhibitor, see e.g., Arthritis & Rheumatism (1996) Vol. 39, No. 9 (supplement), S282), prostaglandin El (see e.g., Arthritis & Rheumatism (1996) Vol. 39, No. 9 (supplement), S282), Tenidap (non-steroidal anti-inflammatory drug, see e.g., Arthritis & Rheumatism (1996) vol. 39, No. 9 (supplement), S280), Naproxen (non-steroidal anti-inflammatory drug, see e.g., Neuro Report (1996) vol. 7, pp. 1209-1213), Meloxicam (non-steroidal anti-inflammatory drug), Ibuprofen (non-steroidal anti-inflammatory drug), Piroxicam (non-steroidal anti-inflammatory drug), Diclofenac (non-steroidal anti-inflammatory drug), Indomethacin (non-steroidal anti-inflammatory drug), Sulfasalazine (see e.g., Arthritis & Rheumatism (1996) Vol. 39, No. 9 (supplement), S281), Azathioprine (see e.g., Arthritis & Rheumatism (1996) Vol. 39, No. 9 (supplement), S281), zap-70 and/or ick inhibitor (inhibitor of the tyrosine kinase zap-70 or ick), VEGF inhibitor and/or VEGF-R inhibitor (inhibitos of vascular endothelial cell growth factor or vascular endothelial cell growth factor receptor, inhibitors of angiogenesis), corticosteroid anti-inflammatory drugs (e.g., SB203580), gold, penicillamine, chloroquine, hydroxychloroquine, chlorambucil, cyclophosphamide, cyclosporine, total lymphoid irradiation, anti-thymocyte globulin, anti-CD4 antibodies, CD5-toxins, collagen, lobenzarit disodium; Cytokine Regulating Agents (CRAs) HP228 and HP466 (Houghten Pharmaceuticals, Inc.), ICAM-1 antisense phosphorothioate oligodeoxynucleotides (ISIS 2302, Isis Pharmaceuticals, Inc.), soluble complement receptor 1 (TP10, T Cell Sciences, Inc.), prednisone, orgotein, glycosamino-glycan polysulphate, minocycline, marine and botanical lipids (fish and plant seed fatty acids, see e.g., DeLuca et al. (1995) Rheum. Dis. Clin. North Am. 21:759-777), auranofin, phenylbutazone, meclofenamic acid, flufenamic acid, intravenous immune globulin, zileuton, mycophenolic acid (RS-61443), tacrolimus (FK-506), sirolimus (rapamycin), amiprilose (therafectin), cladribine (2-chlorodeoxyadenosine) or azaribine or compounds that stimulate anti-inflammatory cytokine mediated signalling pathways (anti-inflammatory compounds) such as interleukin 4 (IL-4, anti-inflammatory cytokine, DNAX/Schering), interleukin 10 (IL-10, SCH 52000, recombinant IL-10, anti-inflammatory cytokine, DNAX/Schering), interleukin-11 (see e.g., Arthritis & Rheumatism (1996) Vol. 39, No. 9 (supplement), S296), interleukin-13 (see e.g., Arthritis & Rheumatism (1996) Vol. 39, No. 9 (supplement), S308) or IL-4-, IL-10-, IL-11 or IL-13 agonists (e.g., agonist antibodies).

Inhibitors are preferred low molecular molecules, antisense molecules or mono or polyclonal antibodies.

More preferred modulators of cytokine mediated signalling pathways are TNFα inhibitors, inhibitors of interleukin-1β converting enzyme (ICE inhibitors) or inhibitors of IL-12 or IL-18, most preferred modulators of cytokine mediated signalling pathways are TNFα inhibitors, particularly TNFα antibodies.

Preferred ICE Inhibitors within the scope of the invention are compounds which have a Kᵢ value of 1 μM or less. Most preferred are those ICE Inhibitors which have a Kᵢ value of 100 nM or less and mostly preferred are those ICE Inhibitors which have a Kᵢ value of 10 nM or less.

Suitable for the combination therapy of the invention are in principle all ICE inhibitors, for example such as L-Alaninamide (N-((phenylmethoxy)carbonyl)-L-valyl-N-((1S)-3-((2,6-dichloroben-zoyl)oxy)-1-(2-ethoxy-2-oxoethyl)-2-oxopropyl), SDZ-224-015, VE-13045, Novartis); 6a,12a-epoxy-1,2,3,4,6a,7,12,12a-octahydro-3,7-dihy-droxy-8-methoxy-3-methyl-benz(a)anthracen-1,12-dione (E1-1507-1, E1-1507-2, Kyowa Hakko), VX-740, HMR-3480 (Aventis, Pharmaprojects databases), N-(N-((2S,3S)-3-trans-carboxyoxirane-2-carbonyl)-L-phenylalanyl)-1,4-diaminobutane (TAN-1756A, TAN-1756B, Ta-45 keda), (2S-cis)-5-(Benzyloxycarbonylamino-1,2,4,5,6,7-hexahy-dro-4-(oxoazepino(3,2,1-hi)indole-2-carbonyl)-amino)-4-oxobutanoic acid, Idun (US).

Suitable for the combination therapy of the invention are in principle all TNFα inhibitors, such as TNFα antibodies, TNFα-convertase inhibitors or the compounds SR-31747 (Cyclohexanamine, N-(3-(3-chloro-4-cyclohexylphenyl)-2-propenyl)-N-ethyl-,hydrochloride, (Z)-(CAS), Sanofi-Synthelabo , Pharmaprojects databank),75 kdTNFR-IgG (75 kD TNF receptor-IgG fusion protein, Immunex; see e.g., Arthritis & Rheumatism (1994) Vol. 37, S295; J. Invest. Med. (1996) Vol. 44, 235A), 55 kdTNFR-IgG (55 kD TNF receptor-IgG fusion protein; Hoffmann-LaRoche), TNF-bp/s-TNFR (soluble TNF binding protein; see e.g., Arthritis & Rheumatism (1996) Vol. 39, No. 9 (supplement).

More preferred TNFα inhibitors are TNFα antibodies, for example as described in EP 186833 B1, EP 614984, EP 516785, EP 626389, EP 492488, EP 351789, EP 659766, WO 9429347, EP 701571, EP 486526, WO 9216553, EP 610201, EP 366043, U.S. Pat. No. 5,672,347, U.S. Pat. No. 5,795,967, U.S. Pat. No. 5,807,715, EP 260610 B1 or WO 9729131.

Most preferred TNFα antibodies are poly- or monoclonal, human, humanized, murine or chimeric TNFα antibodies such as CDP-571/Bay-10-3356 (humanized TNFα antibodiy, Celltech/Bayer), cA2 (chimeric TNFα antibody, Centocor), S284; Amer. J. Physiol.—Heart and Circulatory Physiology (1995) Vol. 268, pp. 37-42), D2E7 (WO 9729131, Knoll AG), MAK 195 (EP 260610, BASF Aktienge-sellschaft), Synergen (AmgenWorld, Scrip 1997, 2216, 26), Yeda (Ares-Serono, Scrip 1992, 1687, 24), BB-2983 (Glaxo Wellcome, Pharmaprojects database), AGT1 (Advanced Biotherapy Concepts), sTNF-R1 (Amgen, Scrip Daily Online, 22 Nov. 1999) or TNF-484 (Novartis, Pharmaprojects database), particularly D2E7.

Further preferred TNFα antibodies are antibodies, or an antigen-binding portion thereof, that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. More preferably, the antibody, or antigen-binding portion thereof, dissociates from human TNFα with a K_{off} of 5×10⁻⁴ s⁻¹ or less, or even more preferably, with a K_{off} of 1×10⁻⁴ s⁻¹ or less. More preferably, the antibodies, or antigen-binding portion thereof, neutralizes human TNFα cytotoxicity in a standard in vitro L929 assay with an IC₅₀ of 1×10⁻⁸ M or less, even more preferably with an IC₅₀ of 1×10⁻⁹ M or less and still more preferably with an IC₅₀ of 5×10⁻¹⁰ M or less.

A “neutralizing antibody”, as used herein (or an “antibody that neutralized hTNFα activity”), is intended to refer to an antibody whose binding to hTNFα results in inhibition of the biological activity of hTNFα. This inhibition of the biological activity of hTNFα can be assessed by measuring one or more indicators of hTNFα biological activity, such as hTNFα-induced cytotoxicity (either in vitro or in vivo), hTNFα-induced cellular activation and hTNFα binding to hTNFα receptors. These indicators of hTNFα biological activity can be assessed by one or more of several standard in vitro or in vivo assays known in the art. Preferably, the ability of an antibody to neutralize hTNFα activity is assessed by inhibition of hTNFα-induced cytotoxicity of L929 cells.

As an additional or alternative parameter of hTNFα activity, the ability of an antibody to inhibit hTNFα-induced expression of ELAM-1 on HUVEC, as a measure of hTNFα-induced cellular activation, can be assessed.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 1 and Jöns-son, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

Preferred integrin αᵥβ₃ receptor antagonists within the scope of the invention are substances which show an IC₅₀ value of 100 nM or less for the inhibition of vitronectin binding to integrin αᵥβ₃ in an ELISA assay, which is, described for example in DE 19919218.9 (German application number).

Suitable integrin αᵥβ₃ receptor antagonists for the combination therapy of the invention are, in principle, all integrin αᵥβ₃ receptor antagonists, for example as described in Pitts et al.; J. Med. Chem. 2000, 43, 27-40; Batt et al., J. Med. Chem. 2000, 43, 41-51; Miller et al., Bioorg. Med. Chem. Lett. 9, 1999, 1807-1812; Keenan et al., Bioorg. Med. Chem. Lett. 9, 1999, 1801-1806; Rockwell et al., Bioorg. Med. Chem. Lett. 9, 1999, 937-942; Samanen et al., Current Pharm. Design 1997, 3, 545-584; Miller et al., J. Med. Chem. 2000, 43, 22-26; Hartmann and Duggan, Exp. Opin. Invest. Drugs 2000, 9 (6), 1281-1291; Miller et al., Drug Discovery Today 2000, 5 (9), 397-408; DE 19919218.9 (German application number), DE 19948269.1 (German application number), DE 19962998.6 (German application number), DE 10027514.1 (German application number), DE 10028575.9 (German application number), DE 10039998.3 (German application number), WO 9952879, WO 9835917, WO 0000486, WO 0017197, WO 0031067, WO 9843962, WO 9926945, WO 9950249, WO 9958162, WO 0000481, U.S. Pat. No. 6,056,958, WO 43787, WO 9637492, WO 9723480, WO 9733887, WO 9748395, WO 9748444, WO 9823608, U.S. Pat. No. 5,849,736, DE 19626701, EP 0796855A1, DE 19653645, DE 19653646, DE 19653647, EP 796855, EP 820988, EP 820991, EP 853084, EP 854145, U.S. Pat. No. 5,990,145, WO 9915506, WO 9915507, WO 9932457, WO 9937621, WO 9959992, EP 928790, EP 928793, U.S. Pat. No. 6,001,855, WO 00024724, WO 9825892, WO 9965944, WO 0048603, WO 9938849, WO 9952872, DE 19534016, DE 19548709, DE 19653036, DE 19654483, DE 19705450, DE 1971300, DE 19725368, DE 19842415, DE 19850131, EP 683173, EP 710657, EP 741133, EP 771 818, WO 9714716, WO 9723451, WO 9738009, WO 9744333, WO 9800395, WO 9818764, WO 9827112, WO 9835949, WO 9901472, WO 9910371, WO 9931126, WO 0003973, WO 0026212, WO 9532710, WO 9726250, WO 9737655, WO 9808518, WO 9808840, WO 9818460, WO 9818461, WO 9831359, WO 9844797, WO 9846220, WO 9901472, WO 9930709, WO 9930713, WO 9931061, WO 9931099, WO 0006169, WO 0009503, U.S. Pat. No. 5,981,546, U.S. Pat. No. 6,017,925, U.S. Pat. No. 6,017,926, WO 9967230, WO 9734865, FR 2768734-A1,FR 2768736-A1, WO 0032578, U.S. Pat. No. 5,639,765, U.S. Pat. No. 5,681,820, U.S. 5,852,210, U.S. Pat. No. 5,972,986, U.S. Pat. No. 6,013,651, WO 9708145, WO 9736858, WO 9736859, WO 9736860, WO 9736861, WO 9736862, WO 9944985, WO 9944994, WO 9951638, WO 9952896, WO 0009143, WO 0038665, WO 0038715, WO 0038719, WO 0038786, WO 9600574, WO 9600730, WO 9606087, WO 9626190, WO 9701540, WO 9724119, WO 9724122, WO 9724124, WO 9724336, WO 9814192, WO 9815278, WO 9829561, WO 9830542, WO 9840488, WO 9905107, WO 9906049, WO 9911626, WO 9915170, WO 9915178, WO 9915508, WO 9945927, WO 0007544, WO 0033838 or WO 9933798, particularly, the following proteins, pepcidic and nonpeptidic compounds.

Proteins and peptidicintegrin αᵥβ₃ receptor antagonists: LM 609 (vitaxin, Pharmaprojects), abciximab (c7E3 Fab, Reopro®, Pharmaprojects), Peptides and peptidomimetics of Arg-Gly-Asp and derivatives thereof like:

cyclo(RGDfV), As-Pen-RGDC-OH, cyclo[RGD-Mamb-P], XJ 735 (cyclo[R-G-D-Mamb-A]), XK 002 (cyclo[(NMe)R-G-D-(2-amino-1,3-thiazol-4yl-acetic acid)-V]), DMP 728 (cyclo[(NMe)R-G-D-Mamb-DAbu]), SK+F 107260

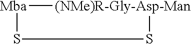

EMD 121974 (cyclo[R-G-D-f-(NMe)V]) and any other RGD containing peptides.

Non-peptidic integrin αᵥβ₃ receptor antagonists: (2R)-2-[((2R)-2-{3-[(3-{[amino(imino)methyl]amino}propanoyl)amino]phenyl}-3-carboxy propanoyl)amino]-3-methylbutanoic acid, 3-[8-(2-{[amino(imino)methyl]amino}ethyl)-1-benzyl-2-oxo-1,2,3,5-tetrahydro-4H-1,4-benzodiazepin-4-yl]propanoic acid, 2,3-dihydroxypropyl 2-{[(benzyloxy)carbonyl]amino}-4-({9, 10-dimethoxy-4-[(E)-2-(1,4,5,6-tetrahydropyrimidin-2-yl)hydrazono]-1,2,3,3a,4,5,6,10b-octahydrobenzo[e]azulen-8-yl}oxy)butanoate, (2S)-2-{[(benzyloxy)carbonyl]amino}-3-[({(4S)-4-[3-(4,5-dihydro-1H-imidazol-2-ylamino)propyl]-2,5-dioxoimidazolidin-1-yl}acetyl)amino]propanoic acid, L-7418415 ((2S)-2-[(phenylsulfonyl)amino]-3-({4-[2-(1,4,5,6-tetrahydropyrimidin-2-ylamino)ethoxy]benzoyl}amino)propanoic acid), (2S)-2-{[(4-isobutylphenyl)sulfonyl]amino}-3-[({5-[3-(pyridin-2-ylamino)propyl]-4,5-dihydroisoxazol-3-yl}carbonyl)amino]propanoic acid, (2S)-2-{[(benzyloxy)carbonyl]amino}-3-[({4-[4-(4,5-dihydro-1H-imidazol-2-ylamino)butanoyl]piperazin-1-yl}carbonyl)amino]propanoic acid, (2S)-2-{[(benzyloxy)carbonyl]amino}-3-[({4-[4-(4,5-dihydro-1H-imidazol-2-ylamino)propanoyl]piperazin-1-yl}carbonyl)amino]propanoic acid, SD-186 ((2S)-2-[(phenylsulfonyl)amino]-3-[{(8-(pyridin-2-ylamino)methyl}-1-oxa-2-aza-spiro[4.5]dec-2-en-3-yl]carbonyl)amino]propionic acid), SD-183 ((2S)-2-[(phenylsulfonyl)amino]-3-[({8-[(pyridin-2-ylamino)methyl]-1-oxa-2-azaspiro[4.5]dec-2-en-3-yl}carbonyl)-amino]propanoic acid, SD-983 ((2S)-2-{[(benzyloxy)carbonyl]amino}-3-[({3-[3-(4,5-dihydro-1H-imidazol-2-ylamino)propoxy]isoxazol-5-yl}carbonyl)amino]propanoic acid), XT-199 ((2S)-3-[({3-[3-(4,5-dihydro-1H-imidazol-2-ylamino)propoxy]isoxazol-5-yl}carbonyl)amino]-2-[(phenylsulfonyl)amino]propanoic acid), SG-545 (Methyl (2S)-2-{[(benzyloxy)carbonyl]amino}-3-[({3-[3-(4,5-dihydro-1H-imidazol-2-ylamino)propoxy]isoxazol-5-yl}carbonyl)amino]propanoic acid), SM 256 ((2S)-3-[({1-[3-(1H-imidazol-2-ylamino)propyl]-1H-indazol-5-yl}carbonyl)amino]-2-[(mesitylsulfonyl)amino]propanoic acid), SD-836 (Pharmaprojects), SD-7784 (Pharmaprojects), SD-7783 (Pharmaprojects), S-137 (N-({[1-(4-{[amino(imino)methyl]amino}butyl)vinyl]amino}acetyl)-3-pyridin-3-yl-beta-alanine), S-787 (Seattle et al.; 21^{st} Ann. Meet. Amer. Soc. Bone Mineral Res., 30.9.-4.10.1999; SU 410), S 448 (N-{[(3-{[amino(imino)methyl]amino}benzoyl)amino]acetyl}-3-phenyl-β-alanine), SC 68448 (N-{[(3-{[amino(imino)methyl]amino}benzoyl)amino]acetyl}-3-(3,5-dichlorophenyl)-β-alanine), SC 56631 (N-{[(5-{[amino(imino)methyl]amino}pentanoyl)amino]acetyl}-3-pyridin-3yl-β-alanine), SC 69000 (4-[(3-{[amino(imino)methyl]amino}benzoyl)amino]-N-(isobutoxycarbonyl)phenylalanine), SC-65811 (N-{[(3-{[(benzylamino)carbonyl]amino}benzoyl)amino]acetyl}-3-pyridin-3-yl-b-alanine), SB 223245 (((2S)-7-{[(1H-benzimidazol-2-ylmethyl) (methyl)amino]carbonyl}-4-methyl-3-oxo-2,3,4,5-tetrahydro-1H-1,4-benzodiazepin-2-yl)acetic acid), SB 265123 ([(10S)-3-[3-(pyridin-2yl-amino)propoxy]-10,11-dihydro-5H-dibenzo[a,d]-cyclohepten-10yl]acetic acid), SB 267268 ([(4S)-3-oxo-8-[3-(pyridin-2-ylamino)propoxy]-2-(2,2,2-trifluoroethyl)-2,3,4,5-tetrahydro-1H-2-benzazepin-4-yl]acetic acid), SB 273005 (Lark et al.; 21^{st} Ann. Meet. Amer. Soc. Bone Mineral Res., 30.9.-4.10.1999; SU201), CP-4632 ((2S)-3-[(3-fluoro-4-[4-(1,4,5,6-tetrahydropyrimidin-2ylamino)piperidin-1yl]benzoyl)amino]-2-[(phenylsulfonyl)amino]propanoic acid), (2S)-3-({3-chloro-4-[4-(1,4,5,6-tetrahydropyrimidin-2-yl)piperidin-1-yl]benzoyl}amino)-2-[(phenylsulfonyl)amino]propanoic acid), SH306 (2S)-2-[(mesitylsulfonyl)amino]-3-[({1-[3-(pyridin-2-ylamino)propyl]-1H-indazol-5-yl}carbonyl)amino]propanoic acid, SB 273005 (Lark et al.; 21st Ann. Meet. Amer. Soc. Bone Mineral Res., 30.9.-4.10.1999; SU201) [(4S)-8-{2-[6-(Methylamino)pyridin-2-yl]ethoxy}-3-oxo-2-(2,2,2-trifluoroethyl)-2,3,4,5-tetrahydro-1H-2-benzazepin-4-yl]acetic acid, SC 72115 (3-(5-bromo-3-chloro-2-hydroxyphenyl)-N-({[3-(4,5-dihydro-1H-imidazol-2-ylamino)benzoyl]amino}acetyl)-beta-alanine).

Preferred are non-peptidic antagonists, particularly those which are orally available and integrin aᵥβ₃ receptor antagonists selected from the group:

LM 609 (vitaxin), EMD 121974 (cyclo[R-G-D-f-(NMe)V]), L-7418415 ((2S)-2-[(phenylsulfonyl)amino]-3-({4-[2-(1,4,5,6-tetrahydropyrimidin-2-ylamino)ethoxy]benzoyl}amino)propanoic acid), SB 265123 ([(10S)-3-[3-(pyridin-2yl-amino)propoxy]-10,11-dihydro-5H-dibenzo[a,d]-cyclohepten-10yl]acetic acid), SB 267268 ([(4S)-3-oxo-8-[3-(pyridin-2-ylamino)propoxy]-2-(2,2,2-trifluoroethyl)-2,3,4,5-tetrahydro-1H-2-benzazepin-4-yl]acetic acid), SB 273005 (Lark et al.; 21^{st} Ann. Meet. Amer. Soc. Bone Mineral Res., 30.9.-4.10.1999; SU201), SC 68448 (N-{[(3-{[amino(imino)methyl]amino}benzoyl)amino]acetyl}-3-(3,5-dichlorophenyl)-β-alanine), SC 69000 (4-[(3-{[amino(imino)methyl]amino}benzoyl)amino]-N-(isobutoxycarbonyl)phenylalanine and SC-65811 (N-{[(3-{[(benzylamino)carbonyl]amino}benzoyl)amino]acetyl}-3-pyridin-3-yl-b-alanine).

All mentioned compounds can also be applied as prodrugs. Prodrugs are substances which metabolise in vivo to the active compound. Examples for such metabolism are first pass metabolisms (e.g. esters to free acids or carboxylates).

“Orally available” means at least 10%, preferred 30% and more preferred 50% for integrin αᵥβ₃ receptor antagonist.

All mentioned compounds may be administered as such or in the form of their salts with physiologically tolerated acids or bases. Antibodies may also be used as antibody-portions.

Preferred combinations of modulators of cytokine mediated signalling pathways with integrin αᵥβ₃ receptor antagonists are selected from the preferred modulators of cytokine mediated signalling pathways and the preferred integrin αᵥβ₃ receptor antagonists.

The modulators of cytokine mediated signalling pathways in combination with the integrin αᵥβ₃ receptor antagonist may be administered together in a pharmaceutical composition or simultaneous via separate ways or separate or temporal graduated.

Therefore, the invention further relates to a pharmaceutical composition, comprising a modulator of cytokine mediated signalling pathways and an integrin αᵥβ₃ receptor antagonist.

This composition can be used as a medicament, particularly for curing or preventing inflammatory or autoimmune disorders, such as rheumatoid arthritis, rheumatoid spondylitis, osteoarthritis, gouty arthritis, allergy, multiple sclerosis, autoimmune diabetes, autoimmune uveitis or nephrotic syndrome.

In a preferred embodiment, the composition is used for the treatment or prevention of rheumatoid arthritis.

The compounds of the invention can be administered orally or parenterally in a conventionally way (subcutaneously, intravenousely, intramusculary, intraperitoneally, rectally). Administration can also take place with vapours or sprays through the nasopharyngeal space. Oral administration is preferred.

The dosage depends on age, condition and weight of the patient and on the mode of administration. The two compounds can be formulated in a single pharmaceutical form or in separate pharmaceutical forms. Administration can be given in several single doses (e.g. 2 to 4) or once or twice a day as depot form.

The weight ratio of integrin αᵥβ₃ receptor antagonist to modulators of cytokine mediated signalling pathways conveniently is in the range of 1:100 to 100:1 preferably 1:10 to 10:1. Advantageously, the dosage to be administered by means of a combination per day and kg amounts to 0,05 to 20 mg of an integrin αᵥβ₃ receptor antagonist and 0,1 to 20 mg, preferably 1 to 10 mg of an modulator of cytokine mediated signalling pathways. In general, the total amount of an integrin αᵥβ₃ receptor antagonist and an modulators of cytokine mediated signalling pathways to be administered daily amounts per kg to a maximum of 50 mg. When a hydrate or a pharmaceutically usable salt is used, then the above values are to be appropriately adjusted.

The compounds can be used individually or together in conventional solid or liquid pharmaceutical forms, e.g. as uncoated or (film-)coated tablets, capsules, powders, granules, suppositories, solutions, ointments, creams or sprays. These are produced in a conventional way. In these, the active substances can be processed with conventional pharmaceutical aids such as tablet binders, fillers, preservatives, tablet disintegrants, flow regulators, plasticizers, wetting agents, dispersants, emulsifiers, solvents, release slowing agents, antioxidants and/or propellant gases (cf. H. Sucker et al. Pharmaceutische Technologie, Thieme Verlag, Stuttgart, 1978). The administration form obtained in this way normally comprises the active substance in an amount of from 0.1% to 99% by weight.

Treatment of a patient with an inflammatory or autoimmune disease by a combination, composition and method according to the present invention may include concomitant use of further adjunctive agents, such as antiinflammatory drugs as described above.

Subject of the present invention are also pharmaceutical compositions, comprising an integrin αᵥβ₃ receptor antagonist in an appropriate container and an modulator of cytokine mediated signalling pathways in a separate container to be used according to the above-mentioned administration regiments.

Pharmaceutical packaging units prepared in accordance with the present invention may consist of an appropriate administration form comprising the integrin αᵥβ₃ receptor antagonist, and an appropriate packaging unit comprising the modulator of cytokine mediated signalling pathways. The two active compounds are preferrably present in the packaging unit in two different containers, e.g. tablets. However, depending on the type of active compound, it may also be possible to provide both compounds in a single dosage form. Further, the pharmaceutical packaging units comprise instructions, for example in the form of a package leaflet prescribed for medicaments from which it follows that the administration of a therapeutically active amount of the integrin αᵥβ₃ receptor antagonist advantageously takes place in combination with administration of an modulators of cytokine mediated signalling pathways.

If applied separately, the administration of the modulators of cytokine mediated signalling pathways takes places before, simultaneously or after the administration of the integrin αᵥβ₃ receptor antagonist.

Information regarding the manner of use can either be given in the information leaflet or as a packing overprint on the medical preparation which can be bought together with medicinal preparations which comprise integrin αᵥβ₃ receptor antagonists. On the one hand, pharmaceutical packaging units comprising only appropriate administration forms of the integrin αᵥβ₃ receptor antagonists can comprise such information e.g. in the form of package leaflets, wherein the combined administration together with modulators of cytokine mediated signalling pathwayss according to the present invention is mentioned. On the other hand, pharmaceutical packaging units comprising only modulators of cytokine mediated signalling pathways can comprise such information wherein the combined administration together with integrin αᵥβ₃ receptor antagonists and the use according to the present invention is mentioned. A third alternative would be to provide pharmaceutical pakkaging units comprising an integrin αᵥβ₃ receptor antagonist, modulators of cytokine mediated signalling pathways and an appropriate information about the combined use of both, e.g. the usual package leaflet.

Therefore, the invention further relates to a pharmaceutical trade package, comprising as pharmaceutical agent an modulator of cytokine mediated signalling pathways or/and an integrin αᵥβ₃ receptor antagonist together with an instruction for use of this pharmaceutical agents in combination for simultaneous, separate, or temporal graduated application for the treatment or prevention of diseases.

Appropriate directions of use of the above-mentioned pharmaceutical agents are essential for commercialization of such pharmaceutical packages, comprising either the integrin αᵥβ₃ receptor antagonist, the modulator of cytokine mediated signalling pathways or a combination thereof.

Commercialization of appropriate pharmaceuticals by pharmaceutical companies is only possible when prior approval of such pharmaceutical agents and the respective administration regimens is achieved by the respective national Health Authorities, such as the FDA in the US or the CPMP Authority in Europe.

This includes but is not limited to performing clinical trials according to well-established procedures under the supervision of said-pharmaceutical company which lateron intends to commercialize such pharmaceutical agents. This also includes filing of appropriate documentation about the results of such clinical trials with the respective Health Authority in order to get marketing approval. The approval is in many cases restricted to certain administration protocols or regimens which have to be included in printed form in the accompanying information leaflet prescribed for medicaments.

### EXAMPLES

### Example 1

### Polyarthitis Model in Tg197 Transgenic Mice

Transgenic mice (Tg197), which have been shown to express human wild type TNFα (modified in the 3′ region beyond coding sequences) develop chronic polyarthritis with 100% incidence at 4-7 weeks of age (See WO 9729131, Example DIII). Transgenic mice are first identified by PCR at 3 days of age and then are verified by by slot blot hybridization analysis at 15 days of age. From the first week of age, litters of transgenic mice are divided into groups of 8 animals each. Before the first weekly injection, average body weight are determined by weighing, all animals in each group and calculating the average body weight. The date and weights of all animals in each group are recorded once a week in the log book.

Each group receive one i.p. injection of a TNFα inhibitor, for example a TNFα antibody, for example D2E7 (see WO 9729131) (dose range 0.1-10 μg/g) or per week or a oad dose (i.v., s.c. or oral) of an integrin αᵥβ₃ antagonist or a combination of both compounds/administrations or vehicle.

The treatment protocols for the six groups are as follows:
Group 1=no treatment;
Group 2=saline (vehicle);
Group 3=TNFα inhibitor, for example a TNFα antibody, for example D2E7 ;
Group 4=integrin αᵥβ₃ antagonist;
Group 5, 6=TNFα inhibitor, for example a TNFα antibody, for example D2E7 in combination with integrin αᵥβ₃ antagonist in different dosages;

A litter with non transgenic mice is also included in the study to serve as a control (Group 7—nontransgenic; no treatment)

Macroscopic changes (in units of arthritic scores) in joint morphology are recorded weekly for each animal. Arthritic scores were recorded as follows; 0=No arthritis, (normal appearance and flexion); +=mild arthritis joint distortion); ++=moderate arthritis (swelling, joint deformation) and +++=heavy arthritis (ankylosis detected on flexion and severely impaired movement).

Sera are collected from 4 out of 8 mice per group by orbital sinus bleeding at 5 weeks of age. At completion of the study all animals are sacrificed and sera are collected by cardiac puncture and stored at −70° C.

Treatment is continued for 8 weeks. At 9 weeks of age, all mice are sacrificed and ankle joints are collected in formalin. Ankle joint sections were then stained with haematoxylin/eosin and histopathology scores are evaluated microscopically in a series of sections. Histopathological scoring based on haematoxylin/eosin staining of joint sections is based as follows; 0=No detectable disease; 1 proliferation of the synovial membrane; 2=heavy synovial thickening, 3=cartilage destruction and bone erosion.

Levels of integrin αᵥβ₃ antagonists are determined by HPLC. Levels of TNFα inhibitor, for example a TNFα antibody, for example D2E7 are determined by EIA according to the validated PK assay (MPF/EB 9644) with one modification. Biotinylated MAX195F is used instead of biotinylated D2E7 in order to eliminate the interference from murine anti-human antibodies. Levels of murine anti human antibodies (MAHA) are determined in a direct ELISA. Microtiter plates were coated with 10 μg/ml of LU 200134 overnight at 4° C., and blocked with 3% teleostean gelatin (Sigma, Cat # G7765) for 2 hours at 25° C. Diluted serum samples or a standard mouse anti-human antibody (Sigma, Cat # M-9035) are added to the plates and incubated overnight at 4° C. Biotinylated D2E7 at 5 nM is added and incubated for 2 hours at 4° C. Plates are washed 5 times with PBS between each step. Avidin coupled alkaline phosphatase (Boehringer Mannheim) are added at 115000 dilution and incubated for 1 hour at 4° C. Bound avidin-alkaline phosphatase is measured with an enzyme amplification kit (TMB, Pierce, Cat # 1854050) according to manufacturer's instructions. ODs are recorded at 490 nm, and the levels of MAHA are assigned from the standard curve.

Statistics: Weekly measurements of weight and ankle joint sizes are recorded for each animal in every group as Excel worksheets. Groups 1 and 7 are compared separately with other groups by two-tailed Student's t-Test. Group 1 and group 7 represent the untreated disease and untreated disease-free control animals, respectively. t-Test function in the Microsoft Excel software was used to obtain probability (P) values of similarity between two groups of experimental animals. The option of two-sample unequal variance was chosen in the t-Test function.

ED₅₀ calculations: For each week, means and standard errors of arthritic scores are plotted as a function of dose. ED₅₀ values are calculated with a non-linear four parameter curve fitting. Histopathological scores determined after the mice have been sacrificed at week 9 are also plotted as a function of dose and ED₅₀ value is derived similarly.

The use of the combination of a modulator of cytokine mediated signalling pathways and an integrin αᵥβ₃ receptor antagonists achieves an inhibitory effect on inflammatory pathomechanisms causing rheumatoid arthritis significantly more pronounced than one of the two treatments alone at the given doses. The combination of a modulator of cytokine mediated signalling pathways and an integrin αᵥβ₃ receptor antagonists in doses too low to be effective alone is effective as a high mono-therapy with either agent and has less potential for side-effects than one principle alone.

1. The method of modulators of cytokine mediated signalling pathways in combination with integrin αᵥβ₃ receptor antagonists for the manufacture of medicaments for the treatment or prevention of diseases.

2. The method of claim 1 for the treatment or prevention of inflammatory or autoimmune disorders.

3. The method of claim 2 for the treatment or prevention of rheumatoid arthritis.

4. The method of claim 1 wherein the modulator of cytokine mediated signalling pathways is a TNFα inhibitor.

5. A pharmaceutical composition, comprising a modulator of cytokine mediated signalling pathways and an integrin αᵥβ₃ receptor antagonist.

6. The composition of claim 5 for use as a medicament.

7. The composition of claim 6 for curing inflammatory or autoimmune disorders.

8. The method of claim 4 for the manufacture of a medicament for the treatment or prevention of diseases.

9. The method of claim 8 for the treatment or prevention of inflammatory or autoimmune disorders.

10. The method of claim 9 for the treatment or prevention of rheumatoid arthritis.

11. A kit comprising as pharmaceutical agent a modulator of cytokine mediated signalling pathways or/and an integrin αᵥβ₃ receptor antagonist together with an instruction for use 40 of this pharmaceutical agents in combination for simultaneous, separate, or temporal graduated application for the treatment or prevention of diseases.
